(19) United States
(12) Patent Application Publication
(10) Pub. No.: US 2021/0298311 A1
(43) Pub. Date: Sep. 30, 2021

(54) FEED MITIGANT COMPOSITIONS FOR INACTIVATING SWINE VIRUSES AND RELATED METHODS

(71) Applicant: Ralco Nutrition, Inc., Marshall, MN (US)

(72) Inventors: Richard Dale LAMB, Balaton, MN (US); Jessica Elizabeth KOPPIEN-FOX, Marshall, MN (US)

(21) Appl. No.: 17/215,677

(22) Filed: Mar. 29, 2021

Related U.S. Application Data
(60) Provisional application No. 63/138,690, filed on Jan. 18, 2021.
(60) Provisional application No. 63/000,945, filed on Mar. 27, 2020.

(51) Int. Cl. A01N 65/22; A01N 65/24; A01N 65/38

(52) U.S. Cl. CPC A01N 65/22; A01N 65/38; A01N 65/24

(57) ABSTRACT

Embodiments include a feed mitigant composition including an essential oil composition and a larch arabinogalactan component. Embodiments include a method of administering the feed mitigant composition including applying a feed mitigant composition to a contaminated feed to form a feed mixture, wherein the feed mitigant composition includes an essential oil composition and a larch arabinogalactan component and wherein the contaminated feed includes feed contaminated with at least one swine virus; and administering the feed mixture to swine, wherein the feed mitigant composition is effective to mitigate transmission of the at least one swine virus from the contaminated feed to the swine.

### BACKGROUND

In 2014, feed was first identified as a risk factor for porcine epidemic diarrhea viral infection of pigs. This observation was expanded across multiple viruses at both the domestic and transboundary levels. In an effort to reduce this risk, it would be desirable to develop techniques for negatively affecting viral survival and viral load in swine feed.

### SUMMARY

In one aspects of the invention, a feed mitigant composition for inactivating at least one swine virus, the feed mitigant composition comprising: at least one of an essential oil composition and a dietary fiber component; wherein application of the feed mitigant composition to a contaminated feed or a feed susceptible to contamination is effective to inactivate at least a portion of at least one swine virus which is present in the feed and/or is to be present in the feed prior to being administered to swine.

In one or more further aspects of the invention, a method of administering a feed mitigant composition, the method comprising: applying a feed mitigant composition to a contaminated feed or a feed susceptible to contamination to form a feed mixture, administering the feed mixture to swine, wherein the feed mixture administered to the swine includes at least one active swine virus; and inactivating at least a portion of the at least one swine virus, wherein the feed mitigant composition includes at least one of an essential oil composition and a dietary fiber component.

### DETAILED DESCRIPTION

### Definitions

The terms recited below have been defined as described below. All other terms and phrases in this disclosure shall be construed according to their ordinary meaning as understood by one of skill in the art.

As used herein, “enhancing” in the context of immune responses refers to one or more of enhancing, priming, potentiating, activating, stimulating, augmenting, boosting, and/or amplifying immune responses in subjects.

As used herein, “immune response” refers to any action relating to a host's immune system and can include, for example, an innate immune response and an adaptive immune response. As used herein, the term “innate immune response” can refer to a non-specific immune response. As used herein, the term “adaptive immune response” can refer to a specific immune response.

As used herein, “plants” and “plant derivatives” can refer to any portion of a growing plant, including the roots, stems, stalks, leaves, branches, seeds, flowers, fruits, and the like. For example, cinnamon essential oil can be derived from the leaves or bark of a cinnamon plant.

As used herein, the term “essential oils” refers to aromatic, volatile liquids extracted from plant material. Essential oils are often concentrated hydrophobic liquids containing volatile aroma compounds. Essential oil chemical constituents can fall within general classes, such as terpenes (e.g., p-Cymene, limonene, sabinene, a-pinene, y-terpinene, b-caryophyllene), terpenoids (e.g., citronellal, thymol, carvacrol, carvone, borneol) and phenylpropanoids (e.g., cinnamaldehyde, eugenol, vanillin, safrole). Essential oils can be natural (i.e., derived from plants), or synthetic.

As used herein “cinnamon essential oil” refers to one or more of natural cinnamon oil (i.e., essential oil derived from plants in the Cinnamomum genus), or synthetic cinnamon oil. Synthetic cinnamon essential oil can comprise synthetic cinnamaldehyde. Synthetic cinnamon essential oil can further comprise one or more major constituents of natural cinnamon essential oil. A major constituent is one which comprises at least 1 wt. %, at least 2.5 wt. %, or at least 5 wt. % of a natural essential oil assay.

As used herein “thyme essential oil” refers to one or more of natural thyme oil (i.e., essential oil derived from plants in the Thymus genus), or synthetic thyme oil. Synthetic thyme essential oil can comprise synthetic thymol. Synthetic thyme essential oil can further comprise one or more major constituents of natural thyme essential oil.

As used herein “oregano essential oil” refers to refers to one or more of natural oregano oil (i.e., essential oil derived from plants in the Origanum genus), or synthetic oregano oil. Synthetic oregano essential oil can comprise synthetic carvacrol. Synthetic oregano essential oil can further comprise one or more major constituents of natural oregano essential oil.

As used herein, the term “emulsion” refers to a fine dispersion of droplets of one liquid in which the liquid is not substantially soluble or miscible. An essential oil may be emulsified or substantially emulsified within an aqueous carrier, for example.

As used herein, the term “emulsifier” refers to a substance that stabilizes an emulsion. The emulsifier can utilize physical properties, chemical properties, or utilize both physical and chemical properties to interact with one or more substances of an emulsion. Tannic acid is an example of an emulsifier for essential oils and water.

As used herein, the term “tannin compound” refers to a polyphenolic biomolecule including at least twelve hydroxyl groups and at least five phenyl groups. Tannin compounds include compounds utilizing gallic acid, flavone and phloroglucinol as base units. Tannic acid (C₇₆H₅₂O₄₆) is one form of a tannin compound. Tannic acid can include quercitannic acid and gallotannic acid, for example.

As used herein, “carrier” refers to a substance that physically binds or combines, or chemically binds or combines, with a target or active substance to facilitate the use, storage, or application of the target or active substance. Carriers are often inert materials, but can also include non-inert materials when compatible with the target or active substances. Examples of carriers include, but are not limited to, water for compositions that benefit from a liquid carrier, or diatomaceous earth for compositions that benefit from a solid carrier.

As used herein, “enzymes” refers to one or more biological molecules capable of breaking down cellulosic material. Enzymes include starch, proteins, non-starch polysaccharides, both soluble and insoluble, lignins and those biological molecules that facilitate chemical reactions within plants and animals.

### Discussion

Feed mitigant compositions and related methods are provided for improving swine health. In some embodiments, the feed mitigant compositions disclosed herein are effective to reduce infections in swine to below clinically detectable levels via at least one of two mechanisms. One mechanism includes use of the compositions disclosed herein as feed mitigants to at least mitigate the impact of virally-contaminated feed consumed by swine. For example, the compositions can inactivate one or more viruses present in the feed. Another mechanism includes use of the compositions to enhance the innate and/or adaptive immune responses of swine through action in the gastrointestinal tract. For example, the compositions can accelerate and strengthen swine immunity to the viruses. Examples of viruses include, without limitation, viruses effecting swine classified in the Baltimore Scheme, including viruses effecting swine classified in one or more of the seven genomic groups I, II, III, IV, V, VI, and VII. In some embodiments, the virses effecting swine include at least one virus selected from Baltimore group IV. Specific examples of viruses include, without limitation, one or more of porcine epidemic diarrhea virus (PEDV), porcine reproductive and respiratory syndrome virus (PRRSV), Seneca Valley virus (SVA) or Senecavirus A, and African swine fever virus (ASFV).

The feed mitigant compositions of the present invention may be combined with or applied to a contaminated feed or a feed susceptible to contamination to form a feed mixture. As used herein, feed, whether contaminated or susceptible to contamination, includes any substance administered to swine and includes, for example and without limitation, feed mixtures, feed premixes, feed additives, feed ingredients, complete feeds, feed components, feed base mixes, any component thereof, and any combination thereof. At least one advantage of the feed mitigant compositions is that the formed feed mixtures may then be immediately administered to swine. In other words, unlike conventional practices and compositions, the formed feed mixtures (or any component thereof) of the present disclosure do not require storage for any duration to mitigate risk of infection to the swine from the swine virus. For example, the feed mitigant composition may be effective to inactivate at least a portion of at least one swine virus, either before, during, or after administering the feed mixture to the swine. In this way, the feed mitigant compositions offer significant cost savings in that storage of feed and/or components of feed is no longer necessary to mitigate risk of infection to swine. At least another advantage of the feed mitigant compositions is that the feed mitigant compositions do not degrade any of the components of the feeds and/or feed ingredients (e.g., vitamins, oils, and other ingredients, etc.).

Feed mitigant compositions may include an essential oil composition and a dietary fiber component, such as a larch arabinogalactan component. The feed mitigant composition may include from about 10% to 90% by weight an essential oil composition and from 10% to 90% by weight a dietary fiber component. All weight percentages may be based on a dry (or partially dry) basis and/or may be based on the total weight of the feed mitigant composition. In some embodiments, the feed mitigant composition includes (a) about 10% by weight the essential oil composition and about 90% by weight the dietary fiber component to (b) about 90% by weight the essential oil composition and about 10% by weight the dietary fiber component. For example, in some embodiments, the feed mitigant composition includes at least 10% by weight the essential oil composition, or at least any one of, equal to any one of, or between any two of 10%, 15%, 20%, 25%, 30%, 35%, 40%, 45%, 50%, 55%, 60%, 65%, 70%, 75%, 80%, 85%, and 90% by weight the essential oil composition by weight the essential oil composition by weight the essential oil composition. In some embodiments, the feed mitigant composition includes at least 10% by weight the dietary fiber component, or at least any one of, equal to any one of, or between any two of 10%, 15%, 20%, 25%, 30%, 35%, 40%, 45%, 50%, 55%, 60%, 65%, 70%, 75%, 80%, 85%, and 90% by weight the dietary fiber component. In some embodiments, the feed mitigant composition includes a 60/40 split of the essential oil composition (60 part) and the dietary fiber component (40 part). In some embodiments, the feed mitigant composition includes a 60/40 split of the dietary fiber component (60 part) and the essential oil composition (40 part). In some embodiments, the feed mitigant composition, the essential oil composition, and/or the dietary fiber component are in the form of solid fine particles (dry).

While not wishing to be bound to a theory, it is believed that the feed mitigant compositions may provide at least two mechanisms for inactivating viruses. A first mechanism is believed to include volatile essential oils that may condense and spread throughout a contaminated feed (or a feed susceptible to contamination) and/or feed ingredients and this may cause inactivation of the at least one swine virus. For example, it is believed that the essential oils may disrupt the viral membrane or interfere with viral envelope proteins involved in host cell attachment. This may involve some contribution from the dietary fiber component as well. A second mechanism is believed to include the dietary fiber component as a safeguard for swine immune system which is also effective to inactivate the at least one swine virus. In some embodiments, administering the feed mitigant composition to feed or feed ingredients is effective to inactivate at least a portion of at least one swine virus. In some embodiments, the portion of the at least one swine virus that is inactivated is inactivated while the feed mitigant composition is mixed with the contaminated feed and/or following administration of the feed mixture to swine.

The dietary fiber component may include at least one of the following: a phytonutrient, a polysaccharide, a dietary fiber, a prebiotic fiber, or a water-soluble fermentable fiber. The dietary fiber component may be selected from larch arabinogalactan, arabinogalactan, polydextrose, chitin, psyllium, methylcellulose, hydrolyzed guar, guar, soy polysaccharide, oat bran, pectin, inulin, fructooligosaccharides (FOS), gum arabic, and combinations thereof. Other suitable dietary fiber components include a yucca plant extract, commercially available as Saponix 5000 or BioLiquid 5000. Another example of a suitable dietary fiber component is yucca plant extract, such as extracts from Yucca schidigera.

In some embodiments, the dietary fiber component includes a larch arabinogalactan. The larch arabinogalactan may be a dry solid of any form. In some embodiments, the larch arabinogalactan includes an agglomerated larch arabinogalactan and/or a non-agglomerated larch arabinogalactan. The larch arabinogalactan can generally include any composition comprising arabinogalactan and optionally other species, such as polyphenols. The larch arabinogalactan can be extracted or derived from any species in the genus Larix. For example, species of the genus Larix include, but are not limited to, Larix laricina, Larix lyallii, Larix leptolepis, Larix occidentalis, Larix decidua, Larix dahurica, Larix sibirica, Larix gmelinii, Larix kaempferi, Larix czekanowskii, Larix potaninii, Larix mastersiana, Larix griffithii, and hybrids thereof. The larch arabinogalactan is available from commercial sources. It can be provided in solid form, such as in the form of a powder, or it can be provided in liquid form, or the solid form can be dissolved to afford a liquid form thereof.

The arabinogalactan can be characterized as a water-soluble, highly or densely branched polysaccharide. The arabinogalactan can generally include any compound composed of galactose units and arabinose units in an approximate ratio of about 100:1 to about 1:1. For example, the arabinogalactan can have a galactan backbone with side chains containing galactose units and arabinose units, wherein a ratio of the galactose units to arabinose units is about 6:1 or about 7.5:1. In an embodiment, the arabinogalactan can be characterized as having a backbone of (1→3)-linked β-D-galactopyranosyl units, each of which can bear a substituent at the C6 position. Most of these side chains can be galactobiosyl units containing a (1→6)-β-D-linkage and α-L-arabinofuranosyl units. These shall not be limiting, as the arabinogalactan can also include arabinogalactan derivatives, such as lipidated and/or quaternized forms of arabinogalactan.

The arabinogalactan can vary in molecular weight from low molecular weight polymers to large macromolecules. The molecular weight of the arabinogalactan can range from about 1,000 Daltons to about 2,500,000 Daltons, or any increment thereof. For example, the molecular weight of the arabinogalactan can range from about 6,000 Daltons to about 2,500,000 Daltons, about 6,000 Daltons to about 300,000 Daltons, about 3,000 Dalton to about 120,000 Dalton, about 15,000 Dalton to about 60,000 Dalton, or about 40,000 Dalton to about 60,000 Dalton, among other ranges.

The larch arabinogalactan can include other species. For example, typically, the larch arabinogalactan comprises polyphenols. The polyphenols can include any compound having two or more phenol groups or moieties. Examples of polyphenols include, but are not limited to, one or more of flavonoids, aromadendrines, anthocyanins, catecholins, catechins, and taxifolins. In an embodiment, the polyphenols include at least flavonoids, such as quercetin. The larch arabinogalactan typically comprises about 1 wt % to about 4 wt % of polyphenols; however, higher and lower concentrations are possible and within the scope of the present disclosure.

The essential oil compositions may include at least one essential oil. In some embodiments, the amount of the at least one essential of the essential oil composition may be at least about 1 ppm, or at least any one of, equal to any one of, or between any two of 1 ppm 10 ppm, 50 ppm 100 ppm, 500 ppm, 1000 ppm, 5000 ppm, 10,000 ppm, and 20,000 ppm or greater. The at least one essential oil may include at least one of a natural essential oil and a synthetic essential oil, such as a nature's equivalent essential oil. The at least one essential oil may be emulsified or may not be emulsified. The essential oil compositions may include any combination of the essential oils disclosed herein, or may include a single essential oil. In some embodiments, the essential oil compositions include one or more essential oils which are organic or non-organic or a combination of organic and non-organic.

In some embodiments, the essential oil compositions include at least one of the following essential oils: oregano essential oil, cinnamon essential oil, thyme essential oil, bergamot essential oil, turmeric essential oil, basal essential oil, lemon grass essential oil, capsicum essential oil, or any combination thereof. In some embodiments, the essential oil compositions include at least one of lemongrass essential oil, basil essential oil, bergamot essential oil, and turmeric essential oil. In some embodiments, the essential oil compositions include at least one of cinnamaldehyde essential oil, oregano essential oil, thyme essential oil, mustard essential oil, cassia essential oil, clove essential oil, rosemary essential oil, turmeric essential oil, nutmeg essential oil, spearmint essential oil, peppermint essential oil, palmarosa essential oil, lavender essential oil, juniperberry essential oil, coriander essential oil, eucalyptus essential oil, anise star essential oil, and caraway essential oil.

In some embodiments, the essential oil composition includes at least one of the following essential oils: cinnamon essential oil, thyme essential oil, oregano essential oil, capsicum essential oil, alfalfa essential oil, allspice essential oil, almond essential oil, ambrette essential oil, angelica root essential oil, angelica seed essential oil, angelica stem essential oil, angostura essential oil, anise essential oil, asafetida essential oil, balm essential oil, balsam of Peru essential oil, basic essential oil, bay leaves essential oil, bay (myrcia oil) essential oil, bergamot (bergamot orange) essential oil, bitter almond essential oil, bois de rose essential oil, cacao essential oil, chamomile flowers essential oil, Cananga essential oil, caraway essential oil, cardamom seed essential oil, carob bean essential oil, carrot essential oil, cascarilla bark essential oil, cassia bark essential oil, celery seed essential oil, cherry essential oil, chervil essential oil, chicory essential oil, citronella essential oil, citrus peels essential oil, clary essential oil, clove bud essential oil, clove leaf essential oil, clove stem essential oil, clover essential oil, coca essential oil, coffee essential oil, cola nut essential oil, coriander essential oil, corn silk essential oil, cumin essential oil, curacao orange peel essential oil, cusparia bark essential oil, dandelion essential oil, dandelion root essential oil, dill essential oil, dog grass essential oil, elder flowers essential oil, estragole essential oil, estragon essential oil, fennel essential oil, fenugreek essential oil, galanga essential oil, garlic essential oil, geranium essential oil, ginger essential oil, glycyrrhiza essential oil, glycyrrhizin essential oil, grapefruit essential oil, guava essential oil, hickory bark essential oil, horehound essential oil, hops essential oil, horsemint essential oil, hyssop essential oil, immortelle essential oil, jasmine essential oil, juniper essential oil, kola nut essential oil, laurel berries essential oil, laurel leaves essential oil, lavender essential oil, lavandin essential oil, lemon essential oil, lemon grass essential oil, lemon peel essential oil, licorice essential oil, lime essential oil, linden flowers essential oil, locust bean essential oil, lupulin essential oil, mace essential oil, malt essential oil, mandarin essential oil, marjoram essential oil, mate 1 essential oil, Melissa essential oil, menthol essential oil, menthyl acetate essential oil, molasses essential oil, mustard essential oil, naringin essential oil, neroli essential oil, nutmeg essential oil, onion essential oil, orange essential oil, origanum essential oil, palmarosa essential oil, paprika essential oil, parsley essential oil, pepper essential oil, peppermint essential oil, peruvian balsam essential oil, petitgrain essential oil, pimento essential oil, pipsissewa leaves essential oil, pomegranate essential oil, prickly ash bark essential oil, rose essential oil, rosemary essential oil, rue essential oil, saffron essential oil, sage essential oil, St. John's bread essential oil, savory essential oil, schinus molle essential oil, sloe berries essential oil, spearmint essential oil, spike lavender essential oil, tamarind essential oil, tangerine essential oil, tannic acid essential oil, tarragon essential oil, tea essential oil, triticum essential oil, vanilla essential oil, violet essential oil, wild cherry bark essential oil, ylang-ylang essential oil, zedoary bark essential oil.

In some embodiments, the one or more essential oils include one or more of cinnamon essential oil, thyme essential oil, oregano essential oil, Cassia redistilled Chinese oil; thyme oil red; origanium; origanium oil organic; thyme white oil; mustard oil; organic oregano; cinnamon bark oil; thyme oil white; allspice leaf oil; thyme oil red organic; cinnamon bark oil organic; clove bud redistilled oil; clove leaf redistilled oil; anise star oil; basil Indian oil; caraway oil; spearmint far west native; rosemary Spanish; lemongrass Indian oil; peppermint Chinese type; palmarosa Indian oil; lavadin grosso maerican; rosemary Moroccan; marjoram sweet oil; rosemary Spanish; rosemary organic; rosemary verbenone organic; lavender spike pure oil; juniperberry oil; petitgrain; eucalyptus glob organic oil; coriander seed oil; balsam peru oil; citronella Java oil; garlic Chinese oil; bergamot BF oil; ginger chinese oil; chamomile roman oil; tangerine; melissa oil; pepper black Indian oil; lime expressed Mexican oil; lemon argentina oil; cardamom oil; carrot seed oil; nutmeg oil; fennel sweet oil; organic turmeric essential oil; grapefruit pink oil; grapefruit white oil; grapefruit red oil; mandarin green Italian oil; mandarin red oil; turmeric essential oil; and oleoresin capsicum.

As mentioned above, in some embodiments, the essential oils can be derived from plants (i.e., “natural” essential oils) and additionally or alternatively their synthetic analogues. Some embodiments comprise a combination of natural and synthetic essential oils. In some embodiments, synthetic essential oils can be a “nature's equivalent” synthetic blend, which generally mimics an essential oil assay of a natural essential oil by including at least 2, at least 5, at least 10, at least 15, at least 20, or any increment thereof, of the most critical essential oils within a natural essential oil. A critical essential oil can be determined by weight percent, and/or by pharmacological efficacy. For example, in one embodiment, a nature's equivalent synthetic oil can comprise the following constitutions as provided in Table 1:

TABLE 1
Nature's Equivalent Synthetic Thyme Essential Oil:

| Constituent | Wt. % |
|---|---|
| Thymol | 42.7-44.08 |
| para-Cymene | 26.88-27.09 |
| Linalool | 4.3-4.34 |
| alpha-Pinene | 4.1-4.26 |
| alpha-Terpineol | 3.14-3.14 |
| 1,8-Cineole | 2.82-3.01 |
| beta-Caryophellene | 1.98-2.27 |
| Limonene | 1.59-1.78 |
| delta-3-Carene | 1.3-1.41 |
| beta-Myrcene | 1.26-1.31 |
| Linalyl Acetate | 1.11-1.24 |
| beta-Pinene | 1.04-1.22 |
| Terpinen-4-ol | 0.96-1.14 |
| alpha-Caryophyllene | 0.71-0.71 |
| gamma-Terpinene | 0.7-0.7 |
| Sabinene | 0.37-0.5 |
| Borneol | 0.27-0.32 |
| Camphene | 0.13-0.17 |

The disclosure herein indicates the efficacy of compositions comprising a plurality of essential oils which provide a synergistic effect beyond essential oils utilized in isolation. Further, the essential oils do not exhibit any antagonistic effects between essential oil moieties within an essential oil composition or feed composition. An essential oil composition can include an essential oil fraction and one or more additional components. The ratio of the essential oil fraction to the one or more additional components in an essential oil composition can depend on several factors such as administration method, and the nutritional/health needs and/or palate of a consuming subject, among others. In some embodiments, a consuming subject comprises animals. In some embodiments, a consuming subject comprises humans. Compositions can comprise additional components including carriers, emulsifiers, and stabilizers, among others. Compositions as provided herein can be in the form of an emulsion. For example, in some embodiments, the feed compositions comprise an essential oil composition, wherein the essential oil composition comprises one or more essential oils and at least one emulsifier, wherein the one or more essential oils are present as an emulsion.

Essential oils suitable for inclusion in the essential oil compositions include any of those disclosed herein. For example, in some embodiments, the essential oil compositions include, without limitation, one or more of cinnamon essential oil, thyme essential oil, oregano essential oil, capsicum essential oil, Cassia redistilled Chinese oil; thyme oil red; origanium; origanium oil organic; thyme white oil; mustard oil; organic oregano; cinnamon bark oil; thyme oil white; allspice leaf oil; thyme oil red organic; cinnamon bark oil organic; clove bud redistilled oil; clove leaf redistilled oil; anise star oil; basil Indian oil; caraway oil; spearmint far west native; rosemary Spanish; lemongrass Indian oil; peppermint Chinese type; palmarosa Indian oil; lavadin grosso maerican; rosemary Moroccan; marjoram sweet oil; rosemary Spanish; rosemary organic; rosemary verbenone organic; lavender spike pure oil; juniperberry oil; petitgrain; eucalyptus glob organic oil; coriander seed oil; balsam peru oil; citronella java oil; garlic Chinese oil; bergamot BF oil; ginger chinese oil; chamomile roman oil; tangerine; melissa oil; pepper black Indian oil; lime expressed Mexican oil; lemon argentina oil; cardamom oil; carrot seed oil; nutmeg oil; fennel sweet oil; organic turmeric essential oil; grapefruit pink oil; grapefruit white oil; grapefruit red oil; mandarin green Italian oil; mandarin red oil; turmeric essential oil; oleoresin capsicum.

In some embodiments, the essential oils include oils from the classes of terpenes, terpenoids, phenylpropenes, or combinations thereof. In some embodiments, the essential oils include oils of plants from the Labiatae or Lamiaceae family, and the Lauraceae family, including hybrids of plants from one or both families Suitable essential oils from the Lauraceae family can comprise those from the Cinnamomum genus. Within the Cinnamomum genus, suitable species can include Cinnamomum burmannii, Cinnamomum cassia, Cinnamomum camphora, Cinnamomum loureiroi, Cinnamomum mercadoi, Cinnamomum oliveri, Cinnamomum osmophloeum, Cinnamomum ovalifolium, Cinnamomum parthenoxylon, Cinnamomum pedunculatum, Cinnamomum subavenium, Cinnamomum tamala, Cinnamomum verum, Cinnamomum verum, and hybrids thereof.

Suitable essential oils from the Lamiaceae family can comprise those from one or more of the Thymus genus, the Origanum genus, the Monarda genus. Within the Thymus genus, a non-limiting list of suitable species can include Thymus caespititius, Thymus capitatus, Thymus carnosus, Thymus citriodorus, Thymus glandulosus, Thymus Herba-borana, Thymus hyemalis, Thymus integer, Thymus pseudolanuginosus (formerly T. lanuginosus), Thymus mastichinia, Thymus montanus, Thymus moroderi, Thymus pannonicus, Thymus praecox, Thymus pulegioides, Thymus serpyllum, Thymus vulgaris, Thymus zygis, and hybrids thereof. Within the Origanum genus, a non-limiting list of suitable species can include Origanum amanum, Origanum compactum, cordifolium, Origanum dictamnus, Origanum laevigatum, Origanum libanoticum, Origanum majorana, Origanum microphyllum, Origanum onites, Origanum rotundifolium, Origanum scabrum, Origanum syriacum, Origanum vulgare, and hybrids thereof. Within the Monarda genus, a non-limiting list of suitable species can include Monarda citriodora, Monarda clinopodioides, Monarda didyma, Monarda fistulosa, Monarda media, Monarda punctata, and hybrids thereof.

Additional examples of essential oils include, without limitation, one or more of the following essential oils: cassia redistilled Chinese oil, thyme oil red, Origanium, natural oregano, origanum oil (organic), thyme white food grade, mustard oil, rosemary essential oil, organic oregano, cinnamon bark oil, thyme oil white, allspice leaf oil, thyme oil red organic, cinnamon bark oil organic, clove bud redistilled oil, clove leaf redistilled oil, anise star oil, basil Indian oil, caraway oil, spearmint far west native, rosemary Spanish oil, lemongrass Indian oil, peppermint Chinese type, palmarosa Indian oil, lavadin grosso American oil, rosemary Moroccan oil, marjoram sweet oil, rosemary organic oil, rosemary verbenone organic oil, lavender spike pure oil, juniperberry oil, petitgrain oil, eucalyptus glob organic oil, coriander seed oil, balsam Peru oil, citronella java oil, garlic Chinese oil, bergamot BF oil, Ginger Chinese Oil, Chamomile Roman Oil, Tangerine oil, Melissa oil, pepper black Indian oil, lime expressed mexican oil, lemon argentina oil, cardamom CO2 oil, carrot seed oil, nutmeg oil, fennel sweet oil, organic turmeric essential oil, grapefruit pink oil, grapefruit whit eoil, grapefruit red oil, mandarin green Italian oil, mandarin red oil, oleoresin capsicum (10⁶) oil, and the like.

In some embodiments, the essential oils can further include lavender essential oils from the Lavandula genus, Mexican bay leaf essential oils from the Liteas genus (e.g., L. glaucescens), West Indian bay tree essential oils from the Pimenta genus (e.g., P. racemosa), Indonesian bay leaf essential oils from the Syzygium genus, bay laurel essential oils from the Laurus genus (e.g., L. nobilis), California bay laurel essential oils from the Umbellularia genus (e.g., U. californica), lemon grass essential oils from the Cymbopogon genus (e.g., C. ambiguous, C. citratus, C. flexuosus, C. martini, C. nardus, C. schoenanthus), spearmint and peppermint essential oils from the Mentha genus (e.g., M. spicata, M. piperita), rosemary essential oils from the Rosmarinus genus (e.g., R. officinalis), sage essential oils from the Salvia genus (e.g., S. sclarea), hybrids thereof, or combinations thereof.

In some embodiments, an essential oil composition can include an essential oil fraction comprising three essential oils from the Lauraceae family and/or the Lamiaceae family In some embodiments, an essential oil composition can include an essential oil fraction comprising cinnamon essential oil from the Cinnamomum genus, thyme essential oil from the Thymus genus, and oregano essential oil the Origanum genus. In some embodiments, an essential oil composition can include an essential oil fraction comprising synthetic cinnamaldehyde and thyme essential oils from the Thymus genus and oregano essential oil from the Origanum genus. In some embodiments, oregano essential oil can comprise carvacrol. Additionally or alternatively, thyme essential oil can comprise thymol.

In some embodiments, the essential oil fraction can comprise about 1% to about 49.5% oregano essential oil, about 1% to about 49.5% thyme essential oil, and about 1% to about 49.5% cinnamon essential oil. In other embodiments, the essential oil fraction can comprise about 5% to about 47.5% oregano essential oil, about 5% to about 47.5% thyme essential oil, and about 5% to about 47.5% cinnamon essential oil. In other embodiments, the essential oil fraction can comprise about 10% to about 45% oregano essential oil, about 10% to about 45% thyme essential oil, and about 10% to about 45% cinnamon essential oil. In other embodiments, the essential oil fraction can comprise about 15% to about 42.5% oregano essential oil, about 15% to about 42.5% thyme essential oil, and about 15% to about 42.5% cinnamon essential oil. In other embodiments, the essential oil fraction can comprise about 20% to about 40% oregano essential oil, about 20% to about 40% thyme essential oil, and about 20% to about 40% cinnamon essential oil. In other embodiments, the essential oil fraction can comprise about 25% to about 37.5% oregano essential oil, about 25% to about 37.5% thyme essential oil, and about 25% to about 37.5% cinnamon essential oil. In other embodiments, the essential oil fraction can comprise about 30% to about 35% oregano essential oil, about 30% to about 35% thyme essential oil, and about 30% to about 35% cinnamon essential oil. In other embodiments, the essential oil fraction can comprise about 33.33% oregano essential oil, about 33.33% thyme essential oil, and about 33.33% cinnamon essential oil.

Many essential oil compositions comprise an essential oil fraction comprising an effective amount of carvacrol, an effective amount of thymol, and an effective amount of cinnamaldehyde. In an essential oil composition including an essential oil fraction comprising oregano essential oil, thyme essential oil, and cinnamon essential oil, the essential oil fraction can comprise three or more natural essential oils wherein the combined essential oils comprise at least an effective amount of carvacrol, at least an effective amount of thymol, and at least an effective amount of cinnamaldehyde. Suitable essential oils can include essential oils from the Cinnamomum genus, essential oils from the Origanum genus, essential oils from the Thymus genus, essential oils from the Monarda genus (e.g., M. citriodora, M. clinopodioides, M. didyma, M. fistulosa, M. media, M. punctata), essential oils from the Trachyspermum genus (e.g., T. ammi), essential oils from the Nigella genus (e.g., N. sativa), and combinations thereof. Other essential oils can be used such that effective amounts of carvacrol, thymol, and cinnamaldehyde are achieved in the essential oil fraction.

In an essential oil composition including an essential oil fraction comprising oregano essential oil, thyme essential oil, and synthetic cinnamaldehyde, the essential oil fraction can comprise two or more natural essential oils and synthetic cinnamaldehyde, wherein the combined essential oils and synthetic cinnamaldehyde comprise at an effective amount of carvacrol, at least an effective amount of thymol, and at least an effective amount of cinnamaldehyde. Suitable essential oils can include essential oils from the Cinnamomum genus, essential oils from the Origanum genus, essential oils from the Thymus genus, essential oils from the Monarda genus (e.g., M. didyma, and M. fistulosa), essential oils from the Trachyspermum genus (e.g., T. ammi), essential oils from the Nigella genus (e.g., N. sativa), and combinations thereof. Still other natural essential oils can be used such that effective amounts of carvacrol, thymol, and cinnamaldehyde are achieved in the essential oil fraction.

In some embodiments, the feed compositions can comprise about 0% to about 100% by weight essential oil(s) or essential oil compound(s). In some embodiments, the weight percent of the essential oil composition, by total weight of the feed composition, is about or up to about 0.1 wt. %, up to about 0.5 wt. %, up to about 1.0 wt. %, up to about 2 wt. %, up to about 3 wt. %, up to about 4 wt. %, up to about 5 wt. %, up to about 6 wt. %, up to about 7 wt. %, up to about 8 wt. %, up to about 9 wt. %, up to about 10 wt. %, up to about 11 wt. %, up to about 12 wt. %, up to about 15 wt. %, up to about 20 wt. %, up to about 22 wt. %, up to about 24 wt. %, up to about 26 wt. %, up to about 28 wt. %, up to about 30 wt. %, up to about 32 wt. %, up to about 34 wt. %, up to about 36 wt. %, up to about 38 wt. %, up to about 40 wt. %, up to about 42 wt. %, up to about 44 wt. %, up to about 46 wt. %, up to about 48 wt. %, up to about 50 wt. %, or any increment or value thereof, or at least one of the aforementioned weight percentages, or no more than the aforementioned weight percentages.

Some essential oil compositions comprise an essential oil fraction comprising an effective amount of carvacrol, an effective amount of thymol, and an effective amount of cinnamaldehyde. An effective amount of carvacrol can comprise at least about 10 wt. %, at least about 15 wt. %, at least about 20 wt. %, at least about 25 wt. %, at least about 30 wt. %, or at least about 33 wt. % of the essential oil fraction. An effective amount of thymol can comprise at least about 10 wt. %, at least about 15 wt. %, at least about 20 wt. %, at least about 25 wt. %, at least about 30 wt. %, or at least about 33 wt. % of the essential oil fraction. An effective amount of cinnamaldehyde can comprise at least about 10 wt. %, at least about 15 wt. %, at least about 20 wt. %, at least about 25 wt. %, at least about 30 wt. %, or at least about 33 wt. % of the essential oil fraction. In some embodiments, oregano essential oil can be replaced by one or more oils which include at least 45 wt. % carvacrol, at least 55 wt. % carvacrol, at least 65 wt. % carvacrol, or at least 75 wt. % carvacrol. In some embodiments, thyme essential oil can be replaced by one or more oils which include at least 30 wt. % thymol, at least 35 wt. % thymol, at least 40 wt. % thymol, or at least 45 wt. % thymol. In some embodiments, cinnamon essential oil can be replaced by one or more oils which include at least 35 wt. % cinnamaldehyde, at least 40 wt. % cinnamaldehyde, at least 50 wt. % cinnamaldehyde, or at least 75 wt. % cinnamaldehyde. Suitable sources of effective amounts of carvacrol, thymol, and/or cinnamaldehyde can include natural essential oils and/or synthetic essential oils.

Essential oil compositions can further comprise one or more of an effective amount of paracymene, an effective amount of eugenol, or an effective amount of citronella. An effective amount of paracymene can comprise at least about 5 wt. %, at least about 7.5 wt. %, at least about 10 wt. %, or at least about 12.5 wt. % of the essential oil fraction. An effective amount of eugenol can comprise at least about 5 wt. %, at least about 7.5 wt. %, at least about 10 wt. %, or at least about 12.5 wt. % of the essential oil fraction. An effective amount of citronella can comprise at least about 5 wt. %, at least about 7.5 wt. %, at least about 10 wt. %, or at least about 12.5 wt. % of the essential oil fraction.

In some embodiments, the essential oil fraction comprises 100% of the essential oil composition. An essential oil composition can further comprise a carrier. Carriers are ideally inert materials which do not react with the active components (i.e., the essential oil fraction) of the composition chemically, or bind the active components physically by adsorption or absorption. Liquid carriers include water, pure water, such as reverse osmosis water, milk, milk replacers, natural and/or commercial liquid feeds, or other liquids germane to animal dietary needs. Milk replacers can be formulated to generally mimic the content of milk. For example, a milk replacer can have a composition similar to that shown in Table 2:

TABLE 2
Example Milk Replacer Formula

| | |
|---|---|
| Crude Protein, minimum | 22.50% |
| Lysine, minimum | 1.60% |
| Crude Fat, minimum | 16.50% |
| Crude Fiber, maximum | 0.50% |
| Calcium, minimum | 0.65% |
| Calcium, maximum | 1.15% |
| Phosphorus, minimum | 0.60% |
| Sodium, minimum | 0.50% |
| Sodium, maximum | 1.00% |
| Selenium, minimum | 0.25 ppm |
| Zinc, minimum | 50 ppm |

The composition can be at least about 50% liquid carrier by weight, at least about 75% liquid carrier by weight, at least about 85% liquid carrier by weight, or at least about 90% liquid carrier. In some embodiments, the composition will be about 80% to about 99% liquid carrier, about 85% to about 98% liquid carrier, about 90% to about 95% liquid carrier, or about 91% to about 94% liquid carrier. In other embodiments, the composition can be about 60% liquid carrier to about 74% liquid carrier, about 63% liquid carrier to about 71% liquid carrier, about 66% liquid carrier to about 68% liquid carrier, or about 67% liquid carrier.

Solid carriers can include limestone, diatomaceous earth, and animal feed. Carriers such as limestone, diatomaceous earth, and the like, are useful pre-feed carriers in that they may be first combined with an essential oil fraction to facilitate transportation and/or subsequent combination of the essential oil composition with a dry carrier such as animal feed. Animal feed can include hay, straw, corn husks, wheat, oats, barley, seeds, commercial livestock feed, and the like. In some embodiments where an essential oil composition comprises an essential oil fraction and a pre-feed carrier, the ratio of pre-feed carrier to the essential oil fraction can be at least 10:1, at least 15:1, at least 17:1, at least 18:1, or at least 20:1. In some embodiments, where an essential oil composition comprises an essential oil fraction and a carrier, with or without a pre-feed carrier, the ratio of carrier to the essential oil fraction can be at least about 1,000:1, at least about 4,500:1, at least about 9,000:1, at least about 20,000:1, at least about 35,000:1, or at least about 50,000:1. In some embodiments, the feed mitigant composition is at least 10% by weight solid carrier(s), or at least any one of, equal to any one of, or between any two of 10%, 15%, 20%, 25%, 30%, 35%, 40%, 45%, 50%, 55%, 60%, 65%, 70%, 75%, 80%, 85%, 90%, and 95% by weight solid carrier(s). For example, in some embodiments, the feed mitigant composition includes about 75% by weight limestone and/or about 15% by weight diatomaceous earth.

The total amount of carrier in a composition can be determined based on the dietary needs of an animal, the tolerance of an animal to essential oil fraction, and other factors. Tolerance can include one or more of an animal's palatability and gastrointestinal tolerance to an essential oil fraction.

An essential oil composition can further comprise one or more dedusting agents. Dedusting agents can comprise vegetable oil, olive oil, mineral oil and the like. The amount of dedusting agent in an essential oil composition can be determined based on the amount required to keep dust low while also allowing a dry composition to be “free flowing”. A suitable “free flowing” characteristic can be determined by a funnel flow test or free flow test. A dedusting agent can be included in a feed in an amount between about 5-40 lbs/ton.

As mentioned above, the essential oil composition may be provided in the form of an emulsion. Accordingly, in some embodiments, the essential oil composition include one or more emulsifiers. In some embodiments, one or more emulsifiers are added to an essential oil composition. An emulsified essential oil fraction can increase the efficacy of an essential oil composition when ingested by a subject, and can make essential oil compositions more palatable to animals which consume the compositions orally. An essential oil fraction can be combined with an emulsifier and a dry carrier, or alternatively an essential oil fraction can be combined with an emulsifier and a liquid carrier, as disclosed above, to form an emulsion. The emulsifier can be combined with an essential oil fraction in a ratio of about 3:1 to about 1:3, about 2:1 to about 1:2, about 1.5:1 to about 1:1.5, or about 1:1. An essential oil composition comprising an essential oil fraction, a liquid carrier, and an emulsifier can have an average essential oil droplet size of less than about 25 microns, less than about 15 microns, less than about 10 microns, less than about 7.5 microns, or less than about 5 microns. In some embodiments, the average droplet size is less than about 7 microns, less than about 6 microns, less than about 5 microns, less than about 4 microns, or less than about 3 microns. As used herein, “droplet size” refers to the average size of an essential oil droplet within an emulsion.

The feed composition and/or essential oil composition can comprise anywhere between about 0% to about 100% by weight emulsifier(s). An emulsifier combined with a liquid carrier can generally be referred to as a liquid emulsifier. In some embodiments, an emulsion can comprise up to about 35%, up to about 40%, up to about 45%, or up to about 50% essential oil fraction and emulsifier, with the balance comprising a liquid carrier. In some embodiments, an emulsion can comprise less than about 20%, less than about 15%, less than about 10%, about 5%, or less than about 5% essential oil fraction and emulsifier, with the balance comprising a liquid carrier. In some embodiments, an emulsion can comprise about 40% to about 60%, or about 45% to about 55% essential oil fraction and emulsifier, with the balance comprising a liquid carrier. In some embodiments, an emulsion can comprise about 1% to about 10%, about 2.5% to about 7.5%, or about 5% essential oil fraction and emulsifier, with the balance comprising a liquid carrier. In many embodiments the liquid carrier is water. The liquid carrier content can vary depending on the amount and type of emulsifier.

One or more emulsifiers can be used to form an emulsion. In some embodiments, one or more emulsifiers can additionally or alternatively be used as a stabilizer. Stabilizers can be used to alter the viscosity of an emulsion. Altering a viscosity can include maintaining a viscosity, increasing a viscosity, or decreasing a viscosity. A suitable emulsifier can be an emulsifier capable of achieving a threshold droplet size. In some embodiments a suitable emulsifier can achieve a suitable emulsion droplet size of less than about 25 microns, less than about 15 microns, less than about 10 microns, less than about 7.5 microns, or less than about 5 microns. In other embodiments, a suitable emulsifier can achieve a suitable emulsion droplet size of less than about 7 microns, less than about 6 microns, less than about 5 microns, less than about 4 microns, or less than about 3 microns. In other embodiments, the emulsion droplet size is in the range of 0.01 microns to about 100 microns, or any increment thereof. An emulsion having a droplet size below a suitable threshold enhances the efficacy of an essential oil composition.

In some embodiments, the feed mitigant composition inactivates the at least one swine virus without a fatty acid. In some embodiments, the feed mitigant compositions are effective without fatty acids. Fatty acids include any type of fatty acid known in the art, including medium chain fatty acids, short chain fatty acids, and long chain fatty acids. In some embodiments, for example, fatty acids include at least one of hexanoic acid, octanoic acid, decanoic acid, and lauric acid. In some embodiments, the essential oil composition is not present as an emulsion. In some embodiments, the feed mitigant composition inactivates the at least one swine virus without formaldehyde. In some embodiments, the feed mitigant composition inactivates the at least one swine virus without an organic acid. In some embodiments, the organic acid includes at least one of the fatty acids described above.

To prepare the feed mitigant compositions disclosed herein, an essential oil composition may be blended or mixed with a dietary fiber component. The essential oil composition may be prepared separate from the dietary fiber component and then subsequently combined with the dietary fiber component, or vice versa. For example, the dietary fiber composition may be prepared separate from the dietary fiber component and then subsequent combined with the essential oil composition.

In some embodiments, the essential oil composition includes at least one of a concentrate and at least one carrier, although other components are permitted (or may be excluded). In some embodiments, the concentrate includes at least one essential oil, which may be an organic or non-organic essential oil, and a liquid, such as a liquid form of a larch arabinogalactan. For example, in some embodiments, the concentrate includes at least one organic or non-organic oregano essential oil, optionally with at least 70% by weight carvacrol, among other phenols. In some embodiments, the concentrate includes pure oregano essential oil, with at least 70% by weight carvacrol, and a liquid larch arabinogalactan. The percentage of the concentrate may include from 10% to 90% of the essential oil and from 10% to 90% of the liquid larch arabinogalactan, including any incremental value or subrange between 10% and 90% for both. In some embodiments, the concentrate includes a 50/50 split of the essential oil and the liquid larch arabinogalactan. In some embodiments, the concentrate is blended with one or more of limestone and diatomaceous earth, one or both of which may sorb the concentrate to obtain a dry or substantially dry essential oil composition. In some embodiments, the concentrate, which may be viscous, is blended with limestone and/or diatomaceous earth until a dry, consistent, finely divided solid that is free flowing is obtained (e.g., solid fine particles).

In some embodiments, a composition for improving swine health comprises a first amount of an oregano essential oil and a second amount of dry larch arabinogalactan. In some embodiments, the oregano essential oil is the only essential oil present in the composition. In some embodiments, the oregano essential oil is emulsified. For example, in some embodiments, the oregano essential oil and liquid larch arabinogalactan are contacted (e.g., mixed, agitated, etc.) under conditions sufficient to form an emulsion, wherein the average particle size of oregano essential oil in the emulsion is about 25 microns or less. In some embodiments, the oregano essential oil is not emulsified. In some embodiments, the second amount of dry larch arabinogalactan is about 40% by weight based on the total weight of the composition. In some embodiments, about 60% of the composition includes at least one carrier, the oregano essential oil, and the liquid larch arabinogalactan. In some embodiments, a method of improving swine health includes contacting the compositions of the present disclosure with a feed and administering said feed to swine. In some embodiments, the compositions of the present disclosure are shown to be efficacious in reducing the risk of infection following consumption of feed contaminated with PRRSV 174, PEDV and SVA.

In some embodiments, the essential oil composition is combined with the dietary fiber component. The dietary fiber component may include one or more of a phytonutrient, a polysaccharide, a dietary fiber, and a water-soluble fermentable fiber. In some embodiments, the dietary fiber component is dry. In some embodiments, the dietary fiber component is a dry larch arabinogalactan. In some embodiments, the dietary fiber component includes from 10% to 90% dry larch arabinogalactan (non-agglomerated) and from 10% to 90% dry larch arabinogalactan (agglomerated). In some embodiments, the dietary fiber component includes a 50/50 split of the dry agglomerated larch arabinogalactan and the dry non-agglomerated larch arabinogalactan. In some embodiments, the mixture of agglomerated and non-agglomerated larch arabinogalactan keeps particles together and improves particle size distribution (e.g., keeps narrower) to prevent separation.

In some embodiments, a composition for improving swine health comprises a first amount of an oregano essential oil and a second amount of dry larch arabinogalactan. In some embodiments, the oregano essential oil is the only essential oil present in the composition. In some embodiments, the oregano essential oil is emulsified. For example, in some embodiments, the oregano essential oil and liquid larch arabinogalactan are contacted (e.g., mixed, agitated, etc.) under conditions sufficient to form an emulsion, wherein the average particle size of oregano essential oil in the emulsion is about 25 microns or less. In some embodiments, the oregano essential oil is not emulsified. In some embodiments, the second amount of dry larch arabinogalactan is about 40% by weight based on the total weight of the composition. In some embodiments, about 60% of the composition includes at least one carrier, the oregano essential oil, and the liquid larch arabinogalactan. In some embodiments, a method of improving swine health includes contacting the compositions of the present disclosure with a feed and administering said feed to swine. In some embodiments, the compositions of the present disclosure are shown to be efficacious in reducing the risk of infection following consumption of feed contaminated with PRRSV 174, PEDV and SVA.

Embodiments of the present disclosure also provide a method of administering the feed mitigant compositions of the present disclosure, the method comprising: applying a feed mitigant composition to a contaminated feed or a feed susceptible to contamination to form a feed mixture, administering the feed mixture to swine, wherein the feed mixture administered to the swine includes at least one active swine virus; and inactivating at least a portion of the at least one swine virus, wherein the feed mitigant composition includes at least one of an essential oil composition and a dietary fiber component. The present embodiment includes all the features of the other embodiments of the present disclosure and thus shall be incorporated by reference herein. In some embodiments, the feed mitigant composition is applied to the contaminated feed at an inclusion rate of between about 0.05% to about 0.3%. In some embodiments, the feed mitigant composition is applied to the contaminated feed or feed susceptible to contamination at a rate between 0.5 to 10 lbs per pound of feed. In some embodiments, the feed mitigant composition is applied at a rate of about 2 lbs per ton of feed. Any portion of the present disclosure provided herein shall be understood to apply to this and any other embodiments of the present disclosure (e.g., even if not repeated).

### Example 1

A study was conducted to evaluate the efficacy of PBCKDRY 3 at reducing the risk of infection and improving the performance and survivability in pigs following consumption of feed contaminated with PRRSV 174, PEDV and SVA. The experimental design consisted of 2 groups: Group 1 included a complete feed mixed with PBCKDRY 3 at a 0.1% inclusion rate; and Group 2 included a complete feed (no mitigants) positive control. The study was based on the hypothesis that differences in the infection events may differ across groups, and utilized a 15-day period with room as the experimental unit with 100 pigs/room (6 pens/room) and a designated feed bin/room. Pigs (15 kg) originated from a source herd documented to be free of all 3 viral pathogens by monthly testing and clinical history. Viral challenge involved a 454 g “Ice Block Model”, consisting of 100 mL SVA (5 logs TCID50/mL, Ct=20.72), 100 mL PRRSV 174 (5 logs TCID50/mL, Ct=21.38), 100 mL PEDV (5 logs TCID50/mL, Ct=24.25) and balanced with 154 mL MEM. Blocks were frozen at −80 degrees C. and dropped into each feed bin on days 0 and 6 of the study. The blocks proceeded to melt, with liquid permeating the feed, which was then augured into room for pigs to consume via natural feeding behavior.

The formulation PBCKDRY 3 may include about 45 wt % limestone, about 9 wt % diatomaceous earth (Natural DE AFA), about 3 wt % pure oregano essential oil, about 3 wt % arabinogalactan liquid (Larafeed), and about 40 wt % arabinogalactan dry (Larafeed). The oregano essential oil may be obtained commercially. Examples of suitable commercial sources include, without limitation, the following: Aprikus Standard 1000 ppm, Aprikus Organic 1000 ppm, Norwest 70% 1000 ppm, Greek 1000 ppm, Carvacrol 1000 ppm, Washington 1000 ppm, and Phoenix organic 1000 ppm.

Ingredient Description

| | | | |
|---|---|---|---|
| Limestone | 10346 | 900 | 0.450 |
| Diatomaceous Earth, Natural DEAFA | 11042 | 180 | 0.090 |
| Pure Oregano Oil, Aprikus (Standard) | 10669 | 60 | 0.030 |
| Arabinogalactan Liquid (LaraFeed) | 10659 | 60 | 0.030 |
| Arabinogalactan Dry (LaraFeed) | 10658 | 800 | 0.400 |
| Total Pounds | | 2000 | 1.000 |

Ante-mortem samples, including oral fluids and Swiffer samples of feeders, were collected across the 6 pens from each of the 2 rooms at 0, 6 and 15 days post-inoculation (DPI) of feed. For collection of feed samples, individual Swiffer cloths were drawn across the feed trough, contacting feed particles present. Cloths were immersed in sterile saline and a 3 mL aliquot was decanted for testing. Post-mortem samples were collected from 30 pigs from each room at 15 DPI. Samples collected included tonsil for SVA, serum for PRRSV and rectal swabs for PEDV. Samples were evaluated for the presence of viral nucleic acid by PCR and nucleic acid sequencing of the ORF 5 was performed on select samples, as needed. In addition, start and end weights were collected from all pigs (0 and 15 DPI), along with mortality. Differences in growth performance between groups were analyzed for significance (p<0.05) using ANOVA. In addition, pigs were scored daily for the presence of the following clinical signs: Lameness/vesicles (SVA); Dyspnea/weight loss/rough hair coat (PRRSV); Diarrhea (PEDV).

All samples collected at 0 DPI were negative for all 3 viruses as determined by PCR. Results of virus detection in feed samples and oral fluid samples, along with summaries of performance, clinical signs and animal infection at 6 and 15 DPI are presented in the tables below:

TABLE 1a
Detection of viral RNA in feed &
oral fluid samples by PCR by pen: DAY 6 PI

| | Feeder samples | | | Oral fluid samples | | |
|---|---|---|---|---|---|---|
| Treatment | PRRSV | PEDV | SVA | PRRSV | PEDV | SVA |
| PBCKDRY | 0/6 (+) | 2/6 (+) | 0/6 (+) | 0/6 (+) | 0/6 (+) | 0/6 (+) |
| 3 | | | | | | |
| (+) control | 2/6 (+) | 4/6 (+) | 3/6 (+) | 6/6 (+) | 4/6 (+) | 3/6 (+) |

TABLE 1b
Ct values (mean and range) detected in feed &
oral fluid samples by pen: DAY 6 PI

| | Feeder samples | | | Oral fluid samples | | |
|---|---|---|---|---|---|---|
| Treatment | PRRSV | PEDV | SVA | PRRSV | PEDV | SVA |
| PBCKDRY | ND | 33 | ND | ND | ND | ND |
| 3 | | (33.2- | | | | |
| | | 33.4) | | | | |
| (+) control | 30.9 | 33.7 | ND | 29.9 | 29.8 | 32.8 |
| | (24.9- | (32.5- | | (25.7-34.5) | (26.3- | (31-35.3) |
| | 36.9) | 34.3) | | | 33.1) | |

TABLE 1c
Detection of viral RNA in feed & oral fluid
samples by PCR by pen: DAY 15 PI

| | Feeder samples | | | Oral fluid samples | | |
|---|---|---|---|---|---|---|
| Treatment | PRRSV | PEDV | SVA | PRRSV | PEDV | SVA |
| PBCKDRY | 3/6 (+) | 3/6 (+) | 3/6 (+) | 2/6 (+) | 1/6 (+) | 2/6 (+) |
| 3 | | | | | | |
| (+) control | 6/6 (+) | 6/6 (+) | 5/6 (+) | 6/6 (+) | 6/6 (+) | 2/6 (+) |

TABLE 1d
Range in Ct values detected in feed & oral
fluid samples by pen: DAY 15 PI

| | Feeder samples | | | Oral fluid samples | | |
|---|---|---|---|---|---|---|
| Treatment | PRRSV | PEDV | SVA | PRRSV | PEDV | SVA |
| PBCKDRY | 26 | 30.7 | 31.9 | 32.4 | 31.5 | 32.9 |
| 3 | (23.5- | (28.7- | (26.5- | (29.3- | | (29.9- |
| | 27.7) | 33.5) | 34.7) | 35.6) | | 35.9) |
| (+) control | 24.5 | 29.2 | 30.9 | 25.9 | 22.3 | 32 |
| | (22.6- | (28.1- | (26.9- | (20.1- | (21.3- | (31-33) |
| | 27.5) | 31.6) | 34.9) | 29.3) | 24.3) | |

TABLE 2
Postmortem & growth performance
measures: DAY 15 PI

| | Infection (pig) | | | Disease (pen) | | | Gain (pen) | |
|---|---|---|---|---|---|---|---|---|
| Treatment | PRRSV | PEDV | SVA | PRRSV | PEDV | SVA | Mortality | ADG |
| PBCKDRY | 0% | 40% | 0% | 0% | 0% | 0% | 0.0% | 1.27^{a} |
| 3 | | | | | | | | |
| (+) control | 100% | 100% | 20% | 100% | 100% | 100% | 6% | 0.54^{b} |

^{a,b}Differences in growth rate determined to be significant at p < 0.05

No evidence of SVA or PRRSV 174 infection was observed in pigs consuming feed treated with PBCKDRY 3. In contrast, evidence of PEDV, SVA and PRRSV 174 infection and clinical signs were observed in the positive control population. In addition, lower mortality and a significant difference in growth was observed in pigs fed PBCKDRY 3 as compared to the positive controls.

The strengths of the study included the use of controlled field conditions involving population size and the use of natural feeding behavior. In addition, a robust 3-way viral challenge model was employed and multiple metrics, such as diagnostics and performance measures, were employed. Limitations included study duration, the potential for a level of challenge that may exceed natural levels in the field, and the fact that only a single dose of each virus was utilized.

Under the conditions of the study, it was determined that the challenge model worked. Feed in the bins was successfully contaminated using the ice block, resulting in delivery of all 3 viruses to all rooms. Transmission of PRRSV, PEDV and SVA via the feed was documented in the positive control group. These data support the risk of contaminated feed as a means of viral entry to farms. In addition, it was determined that mitigation was effective. Pigs fed PBCKDRY 3 remained free of PRRSV 174 and SVA infection and clinical signs were not detected in either room. Despite evidence of PEDV RNA in feces, no evidence of clinical signs of PED were observed. Pigs fed PBCKDRY 3 grew faster than positive controls and fewer animals died. Under the conditions of this study, PBCKDRY 3 thus proved to be beneficial to both pig health and performance

What is claimed is:

1. A method of administering a feed mitigant composition, the method comprising:
applying a feed mitigant composition to a contaminated feed or a feed susceptible to contamination to form a feed mixture,
administering the feed mixture to swine, wherein the feed mixture administered to the swine includes at least one active swine virus; and
inactivating at least a portion of the at least one swine virus, wherein the feed mitigant composition includes at least one of an essential oil composition and a dietary fiber component.

2. The method of claim 1, wherein the contaminated feed and/or the feed susceptible to contamination is or includes at least one feed ingredient.

3. The method of claim 1, wherein the dietary fiber component is a phytonutrient, a polysaccharide, a dietary fiber, or a water-soluble fermentable fiber.

4. The method of claim 1, wherein the dietary fiber component includes a larch arabinogalactan.

5. The method of claim 1, wherein the at least one swine virus is porcine reproductive and respiratory syndrome virus (PRRSV).

6. The method of claim 1, wherein the at least one swine virus is Senecavirus A (SVA).

7. The method of claim 1, wherein the at least one swine virus is porcine epidemic diarrhea virus (PEDV).

8. The method of claim 1, wherein the at least one swine virus is African swine fever virus (ASFV).

9. The method of claim 1, wherein the essential oil composition includes at least 500 ppm of an essential oil.

10. The method of claim 1, wherein the essential oil composition includes oregano essential oil.

11. The method of claim 1, wherein the essential oil composition includes or further includes at least one of the following essential oils: capsicum essential oil, rosemary essential oil, cinnamon essential oil, thyme essential oil, and oregano essential oil.

12. The method of claim 1, wherein the essential oil includes or further includes at least one of lemongrass essential oil, basil essential oil, bergamot essential oil, and turmeric essential oil.

13. The method of claim 1, wherein the essential oil composition is not present as an emulsion.

14. The method of claim 1, wherein the feed mitigant composition inactivates the at least one swine virus without formaldehyde.

15. The method of claim 1, wherein the feed mitigant composition inactivates the at least one swine virus without an organic acid.

16. The method of claim 1, wherein the organic acid includes at least one medium chain fatty acid, and wherein the at least one medium chain fatty acid includes at least one of hexanoic acid, octanoic acid, decanoic acid, and lauric acid.

17. The method of claim 1, wherein the feed mitigant composition is applied to the contaminated feed at an inclusion rate of between about 0.05% to about 0.3%.

18. A feed mitigant composition for inactivating at least one swine virus, the feed mitigant composition comprising:
at least one of an essential oil composition and a dietary fiber component;
wherein application of the feed mitigant composition to a contaminated feed or a feed susceptible to contamination is effective to inactivate at least a portion of at least one swine virus which is present in the feed and/or is to be present in the feed prior to being administered to swine.

19. The feed mitigant composition of claim 18, wherein the feed mitigant composition is effective to inactivate at least one of the following viruses: porcine reproductive and respiratory syndrome virus (PRRSV); Senecavirus A (SVA); porcine epidemic diarrhea virus (PEDV); and African swine fever virus (ASFV).

20. The feed mitigant composition of claim 18, wherein the feed mitigant composition excludes at least one of a fatty acid, a formaldehyde, and an organic acid.
